(19) United States
(12) Patent Application Publication
(10) Pub. No.: US 2017/0191024 A1
(43) Pub. Date: Jul. 6, 2017

(54) METHOD FOR INDUCING THE SYNTHESIS OF PHYCOBILIPROTEINS.

(71) Applicant: ALGOBIOTECH, Evry Cedex (FR)

(72) Inventors: Hatem BEN OUADA, EVRY CEDEX (FR); Jihene AMMAR, EVRY CEDEX (FR)

(73) Assignee: ALGOBIOTECH, Evry Cedex (FR)

(21) Appl. No.: 15/505,935

(22) PCT Filed: Aug. 28, 2015
(86) PCT No.: PCT/FR2015/052294

§ 371 Date: Feb. 23, 2017

(30) Foreign Application Priority Data
Aug. 28, 2014 (FR) 1401919

(51) Int. Cl. C12N 1/12; A23L 33/105; A61K 8/97; A23L 17/60; C12P 21/00; A61K 36/02

(52) U.S. Cl. CPC C12N 1/12; C12P 21/00; A61K 36/02; A61K 8/975; A23L 17/60; A23L 33/105; A23V 2002/00

(57) ABSTRACT

The present invention relates to a method for preparation of a biomass of photosynthetic microalgae having a phycobiliprotein content equal to at least 20% of the dry weight of said biomass. The present invention also relates to a biomass enriched with phycobiliproteins and the use thereof as a food supplement or ingredient for nutraceutical and cosmetic preparations.

### TECHNICAL FIELD

The present disclosure relates to a method for inducing the synthesis of phycobiliproteins from an algal biomass.

More particularly, the present disclosure relates to a method for preparing a biomass of photosynthetic microalgae having a content of phycobiliproteins at least equal to 20% of the dry weight of said biomass, comprising the steps of blocking the growth of said biomass and of inducing the synthesis of phycobiliproteins.

### BACKGROUND

Phycobiliproteins are photosynthetic pigment proteins present in microalgae of the groups of the Rhodophyceae, Cryptophyceae and cyanobacteria (blue-green algae). They are protein pigments having a coloring capacity ranging from red to blue.

Among the phycobiliproteins, a distinction is made between phycocyanin, phycoerythrin, allophycocyanin and phycoerythrocyanin.

Phycocyanin is a natural blue protein pigment found exclusively in blue-green algae: cyanobacteria. Phycocyanin is a component of the electron transfer chain situated in phycobilisomes, the role of which is to collect light energy in the red part of the visible spectrum and transfer it to the reaction center of photosystem II of the photosynthetic complex. In some stress conditions which inhibit photosynthetic activity, phycocyanin participates in the mechanisms of protection which consist in dissipating the excess energy by fluorescence. These conditions lead to an increase in the synthesis of this pigment and the accumulation thereof in the form of a nitrogenase reserve for the cell.

The essential factors which govern the synthesis of phycocyanin in several species of cyanobacteria are the nitrogen and iron content, light and temperature. It has been demonstrated that cyanobacteria may regulate their phycocyanin content depending on these parameters within a variation range unique to each species (Hemlata, 2009).

Phycocyanin has antioxidant and fluorescent properties which enable it to have various pharmaceutical and medical applications (Erikson, 2008 and Liu et al, 2000). Its natural character, its extraction in aqueous solution and in the absence of any organic solvent, in addition to its therapeutic properties and its unique blue color are all advantages which enable it to be easily integrated into the demands of European and American standards, linked to the food and therapy markets.

In addition, solving the problems linked to the stability of phycocyanin in aqueous solution, to its high photosensitivity, and to the difficulty with which it is conserved, has enabled global prosperity of the phycocyanin market over the last few years.

The phycocyanin sold on the global market is extracted, exclusively, from one cyanobacterium: Arthrospira platensis (spirulina). Spirulina is a cyanobacterium produced on a large scale and has the advantage of synthesizing up to 25% of its dry weight in phycocyanin under certain conditions, according to the literature.

Phycocyanin production is generally affected by various environmental factors (Niels T. Eriksen, 2008, Remziye Aysun Kepekçi & Saadet Demirörs Saygideger, 2012). Nitrogen and light, in particular, are the most important factors. The former is necessary for the synthesis of the amino acids which constitute proteins and other cellular compounds including phycocyanins (Samy Boussiba and Amos E. Richmond, 1980 and Remziye Aysun Kepekçi & Saadet Demirörs Saygideger, 2012), while light is the source of energy for all photosynthetic activity.

It is known to those skilled in the art that biomass productivity is linked to the density of the culture by a parabolic relationship passing through an optimum. The optimum density promotes primary metabolism and therefore directing of the energy from photosynthesis towards growth and reproduction. When the density is high, beyond a certain threshold, growth stops and the energy collected by photosynthesis is directed towards secondary metabolism and therefore towards the synthesis of defense (or stress) metabolites. Under certain stress conditions (excess nitrogen, low brightness) phycocyanins constitute the most synthesized defense metabolites (Chen et al., 1996; Tomaselli et al., 1997).

Moreover, in the prior art, the culture conditions making it possible to optimize the synthesis of phycocyanins and those which make it possible to optimize the growth of the biomass are quite opposed. This is because the optimum microalgal growth conditions generally vary from 100 to 200 μmol m⁻² s⁻¹ (3300 to 6600 Lux). Microalgal culture produced at an optimum light intensity will enable rapid growth of the biomass but a fairly low synthesis of phycocyanin. Conversely, microalgal culture produced at lower light intensities (10 to 40 μmol m⁻² s⁻¹) grows slowly and therefore leads to a low productivity of the biomass. However, in these same conditions of low light intensity, the contents of phycocyanins may double or triple in value.

Moreover, the greatest syntheses of phycocyanin are obtained under conditions of excess nitrogen and therefore nitrogen contents which exceed those administered in industrial cultures.

There are various systems for producing microalgae, so-called open, natural systems, in lagoons or lakes, and so-called closed systems, consisting of photobioreactors.

Open systems, commonly shallow lakes (30 cm maximum), are the most frequently used because they are simple to use and not very costly. Nonetheless, they have the disadvantage of being subject to variations in climate conditions and to water evaporation, and the mixing of the culture is often insufficient to enable good exposure to light. They thus have two main drawbacks: the lack of control of the culture and low productivity.

Closed systems in photobioreactors are controlled systems, in terms of water or nutrients. Nonetheless, these are very costly systems, the use of which is not easy, especially as regards access to light.

Throughout the analyses carried out by the inventors on the spirulina powder or on fresh biomass coming from different origins and resulting from different growing cultures, whether in a lake or in a photobioreactor, the maximum phycocyanin content obtained is 12% of the dry weight of the biomass. The contents are variable from one sample to another and are between 3% and 12% of the dry weight of the biomass. The contents of phycobiliproteins obtained are therefore always below the maximum values (20 to 25% of the dry weight) of phycobiliproteins present initially in the biomass; these maximum values are only achievable over long growth periods.

All the prior studies aiming to increase the synthesis of phycocyanins have been carried out during the growth phase. Moreover, throughout the scientific literature concerned with the synthesis of phycocyanin, the various stress factors which promote this synthesis have been studied independently of one another.

In a production process, the factors interact with one another in a synergistic or antagonistic way on the synthesis of phycocyanin, depending on the strength of each factor. Finding the focal point of the stress factors, in a process with multiple factors, is necessary to maximize the synthesis of this metabolite.

### SUMMARY

Consequently, the technical problem which the disclosure seeks to solve is that of optimizing the synthesis of phycobiliproteins from a biomass of photosynthetic microalgae without adversely affecting the growth of said biomass, by defining the optimum conditions of growth of the microalga and of synthesis of phycobiliproteins.

The technical problem is solved by the method as claimed in the present disclosure, which comprises a step of blocking the growth of the biomass and a step of inducing the synthesis of phycobiliproteins, said method being adaptable to any conventional system for culturing microalgae, given that it is carried out after taking off a portion of a microalgal culture.

Thus, the main subject of the present disclosure is a method for preparing a biomass of photosynthetic microalgae, in particular of cyanobacteria, more particularly of Arthrospira platensis, comprising inducing the synthesis of phycobiliproteins in a biomass, the growth of which is blocked. The method of the present disclosure makes it possible to obtain a biomass having a content of phycobiliproteins at least equal to 20% of the dry weight of said biomass.

Said photosynthetic microalgae are in particular chosen from Rhodophyceae, Cryptophyceae and cyanobacteria, more particularly cyanobacteria, even more particularly Arthrospira platensis and Aphanizomenon flos-aquae, especially Klamath alga.

During the induction, said biomass, the growth of which is blocked, is in contact with an induction medium, in particular an induction solution.

The method of the present disclosure makes it possible in particular to obtain a biomass having a content of phycobiliproteins at least equal to 21%, 22%, 23%, 24% or 25% of the dry weight of said biomass.

The method of the present disclosure makes it possible in particular to obtain a biomass having a content of phycobiliproteins of from 20% to 25% of the dry weight of said biomass.

Since the method according to the disclosure is carried out outside of the culture tank, it makes it possible to not adversely affect the productivity of the biomass.

The term “biomass” is intended to mean the total mass of living microalgae at a given moment in time in a culture. It is expressed in g/l.

The term “phycobiliproteins” is intended to mean the water-soluble pigment proteins of photosynthesis, such as allophycocyanin (APC), the family of the phycocyanins (C-PC and R-PC), the family of the phycoerythrins (R-PE and C-PE), and phycoerythrocyanin (PEC), isolated from certain microalgae or cyanobacteria.

The term “inducing synthesis” is intended to mean the stimulation, by any means, of the production of phycobiliproteins by a biomass of microalgae.

The term “induction medium” is intended to mean any medium for culturing microalgae containing one or more salts in excess or in deficit relative to the concentration of salts necessary for optimum growth. Within the context of the disclosure, this medium contains an excess of nitrogen obtained by the addition of NaNO₃ to Zarrouk's culture medium, at a concentration of greater than 2.5 g/l and preferably from 3 to 4 g/l.

The term “blocking the growth” is intended to mean stopping the multiplication of the microalga. Blocking the growth is reversible. This is because, following blocking the growth, a portion of the biomass is collected. The concentration is then thus reduced and the biomass may grow once again. The biomass is therefore not adversely affected.

Blocking the growth (the fact that the multiplication of the microalgae is blocked) is determined, for example, by following the curve of the rate of growth of the microalgae as a function of the culture density. Blocking is observed when the rate of growth of the microalgae is zero.

In embodiments of the disclosure, the method is characterized in that blocking the growth of said biomass of microalgae, in particular of cyanobacteria, more particularly of Arthrospira platensis, is executed in an induction tank, said concentration of said biomass in said tank being from 3 to 13 times higher than the concentration enabling the optimum growth of microalgae in culture.

In embodiments of the disclosure, the method is characterized in that blocking the growth of said biomass of microalgae, in particular of cyanobacteria, more particularly of Arthrospira platensis, is executed in an induction tank, said concentration of said biomass in said tank being 3, 4, 5, 6, 7, 8, 9, 10, 11, 12 or 13 times higher than the concentration enabling the optimum growth of microalgae in culture.

The term “concentration” may be replaced by the term “density”. No distinction is made between references to “biomass density” or “biomass concentration”. The density and the concentration are both expressed in g/l.

It is accepted that the density/concentration enabling the optimum growth of microalgae in culture is approximately 0.4 g/l.

This blocking is carried out within the context of the disclosure by the introduction of the biomass, at a concentration ranging from 1 to 5 g/l, into an induction tank, but this means is nonlimiting and any means of blocking the growth of the biomass may be used within the context of the disclosure. The concentration of said biomass at the moment of blocking may be equal to 1 g/l, 1.5 g/l, 2 g/l, 2.5 g/l, 3 g/l, 3.5 g/l, 4 g/l, 4.5 g/l or else 5 g/l.

The density/concentration of the biomass may be measured by different methods.
Secchi disk: this is a simple device making it possible to quickly estimate the density of the cells cultured in aquatic medium. It consists of a graduated rule 30 cm in length, provided with a white disk 5 cm in diameter at the bottom end, at the zero mark. The depth in centimeters, from which the disk can no longer be seen once submerged in the medium, is noted.
Counting under a microscope. This method is time-consuming, but it makes it possible to estimate the number of filaments and the number of turns per filament. By means of a calibrated pipette, a drop of sample is deposited on a depression slide to observe it under a microscope; the number of filaments in a drop is evaluated, it being known that 17 drops from our pipette represent a volume of 1 ml. If the culture is very concentrated, the sample is diluted. The mean number of turns is calculated over 30 filaments chosen randomly.
The optical density at 665 nm. This method makes it possible to quickly estimate the biomass using the absorbance at 665 nm, which is one of the absorption wavelengths of chlorophyll. This in vivo absorption is generally well correlated to the chlorophyll concentration. A spectrophotometer fitted with a 25 cl vessel with parallel faces is used. Zeroing is carried out on culture medium which has not been inoculated.

The induction tank may be an intermediate culture tank or a recovery tank or any other closed system for storing living microalgae. In embodiments of the disclosure, the concentration of said biomass in the induction tank may be between a value substantially equal to or greater than 1 g/l and a value substantially equal to or less than 5 g/l. This concentration is obtained by adding induction medium into the induction tank.

The concentration from 1 g/l to 5 g/l corresponds to a density from 3 to 13 times greater than the density of the biomass growing at the time of the collection, that is to say at the moment of optimum growth, at which the concentration is approximately 0.4 g/l. At this concentration, growth is blocked to promote the defense metabolism of the microalga.

The growth of a biomass, for example in optimum growth, especially at a concentration of approximately 0.4 g/l, may be blocked by bringing said biomass to a concentration of from 1 g/l to 5 g/l, for example by filtration, decantation or centrifugation.

The biomass may also be subjected to washing before being deposited in the induction tank. The washing makes it possible to remove excess salts originating from the culture medium. This thus enables better control of the nitrogen concentration of the induction medium, in particular of the induction solution.

In this case the biomass is washed 3 times using a physiological water or the freshly prepared induction medium solution.

Under these conditions, the initial biomass is not affected by the method and the induction of the synthesis of the phycocyanins is not dependent on the conditions of culture of the biomass.

This procedure has the advantage of being able to be readily integrated into the production line of a traditional spirulina culture farm. It may be applied in an intermediate culture tank or in a recovery tank for a duration of 3 hours, 4 hours or up to 5 hours.

In embodiments of the disclosure, the step of inducing the synthesis of phycobiliproteins comprises:
1) exposing the biomass of photosynthetic microalgae, in particular of cyanobacteria, more particularly of Arthrospira platensis, to a light flux, said flux having a light intensity of a value from substantially equal to or greater than 10 μmol m⁻² s⁻¹ to a value substantially equal to or less than 13 μmol m⁻² s⁻¹;
2) adding a source of nitrogen so as to obtain a concentration of NaNO₃ in the induction medium of greater than 2.5 g/l.

The light intensity in micromoles of photons per square meter and per second is for example determined by means of a photometer measuring the radiation active in the photosynthesis.

It should be noted that 10 μmol m⁻² s⁻¹ correspond to 330 Lux and that 13 μmol m⁻² s⁻¹ correspond to 429 Lux, in particular when the lighting is a fluorescent tube, for example of the “plant growth fluorescent” type. In this case, 1 μmol of photons per square meter and per second is equal to 33 Lux.

The concentration of NaNO₃ in the induction medium is especially from 3 to 4 g/l or from 3 to 5 g/l.

The combination of a light intensity within a range extending from 10 to 13 μmol m⁻² s⁻¹ and a culture solution containing 3 to 5 g/l of NaNO₃ (as nitrogen source) makes it possible to obtain a biomass containing from 20 to 25% phycocyanin, which is particularly high compared to the range of 3 to 12% obtained in the experiments carried out without applying the method of the disclosure.

The light may be provided by simple 40 W power incandescent light bulbs or by fluorescent tubes.

According to some embodiments, the induction medium may be composed of Zarrouk's culture medium, but any other culture medium suited to spirulina may be used. In any case, the nitrogen concentration of the induction medium, in particular of the induction solution, is modified by adding sodium nitrate (NaNO₃) in excess. The final concentration must be within a range extending from 3 to 5 g/l of NaNO₃, which represents a concentration 1.2 to 2 times greater than that present in the initial Zarrouk's medium (2.5 g/l of NaNO₃).

Under the culture conditions defined above, the induction step is carried out for a duration of 3 hours, 4 hours or up to 5 hours and is sufficient to induce the synthesis of phycobiliproteins at a stable content of between 20 and 25% of the dry biomass.

The method according to the disclosure for preparing a biomass of photosynthetic microalgae, in particular of cyanobacteria, more particularly of Arthrospira platensis, having a content of phycobiliproteins at least equal to 20% of the dry weight of said biomass, comprises a step of blocking the growth of the biomass and a step of inducing the synthesis of phycobiliproteins.

According to embodiments, the biomass may have a content of phycobiliproteins at least equal to 21%, 22%, 23%, 24% or 25% of the dry weight of said biomass.

According to embodiments, the biomass may have a content of phycobiliproteins of from 20% to 25% of the dry weight of said biomass.

In one particular embodiment, the method of the present disclosure may comprise a prior step of collecting a biomass of microalgae, in particular of cyanobacteria, more particularly of Arthrospira platensis. The biomass is collected in customary culture tanks, when the culture is in the exponential growth phase, that is to say when the biomass productivity is at maximum. The volume recovered is filtered through gauze (or by decantation or by centrifugation) to obtain the fresh biomass. The biomass obtained is transferred to the induction tank.

The method according to the disclosure may also comprise a final step of collecting the enriched biomass.

The method according to the disclosure may thus comprise the steps of
a) collecting a biomass of microalgae, in particular of cyanobacteria, more particularly of Arthrospira platensis
b) blocking the growth of said biomass
c) inducing the synthesis of phycobiliproteins
d) collecting said biomass enriched in phycobiliproteins.

The method according to the disclosure uses photosynthetic microalgae chosen from cyanobacteria, rhodophytes or cryptophytes.

The photosynthetic microalga used to carry out the method according to the disclosure is Arthrospira platensis, also referred to as spirulina.

Said photosynthetic microalgae are in particular chosen from Rhodophyceae, Cryptophyceae and cyanobacteria, more particularly cyanobacteria, even more particularly Arthrospira platensis and Aphanizomenon flos-aquae, especially Klamath alga.

The phycobiliproteins synthesized within the context of the present method may be phycocyanin, allophycocyanin, phycoerythrin or phycoerythrocyanin or a mixture of at least two of these phycobiliproteins.

The present disclosure also relates to the biomass of photosynthetic microalgae, in particular of cyanobacteria, more particularly of Arthrospira platensis, having a content of phycobiliproteins at least equal to 20% of the dry weight of said biomass, able to be obtained by the previously described method.

According to one advantageous embodiment the biomass has a content of phycobiliproteins at least equal to 21%, 22%, 23%, 24% or 25% of the dry weight of said biomass.

According to one advantageous embodiment the biomass has a content of phycobiliproteins of from 20% to 25% of the dry weight of said biomass.

More particularly, the biomass of photosynthetic microalgae of the present disclosure is a biomass extracted from Arthrospira platensis algae.

Said photosynthetic microalgae are in particular chosen from Rhodophyceae, Cryptophyceae and cyanobacteria, more particularly cyanobacteria, even more particularly Arthrospira platensis and Aphanizomenon flos-aquae, especially Klamath alga.

The biomass of photosynthetic microalgae, in particular of cyanobacteria, more particularly of Arthrospira platensis, able to be obtained by the method of the present disclosure, may be used as a food supplement within the context of preparation of nutraceutical products or as a constituent of cosmetic preparations.

Nutraceutical product is intended to mean any product manufactured from food substances, but made available in the form of tablet, powder, drink or any other medicinal form not conventionally associated with food, and which has proven to have a beneficial physiological effect or protective effect against diseases.

The present disclosure also relates to a cosmetic or nutraceutical composition or a composition intended as a food supplement, prepared from a biomass or a mixture of biomasses of photosynthetic microalgae, in particular of cyanobacteria, more particularly of Arthrospira platensis having a content of phycobiliproteins at least equal to 20% of the dry weight of said biomass.

According to embodiments, the biomass may have a content of phycobiliproteins at least equal to 21%, 22%, 23%, 24% or 25% of the dry weight of said biomass.

According to embodiments, the biomass may have a content of phycobiliproteins of from 20% to 25% of the dry weight of said biomass.

Said photosynthetic microalgae are in particular chosen from Rhodophyceae, Cryptophyceae and cyanobacteria, more particularly cyanobacteria, even more particularly Arthrospira platensis and Aphanizomenon flos-aquae, especially Klamath alga.

Said cosmetic or nutraceutical composition or composition intended as a food supplement may be prepared from a biomass of Arthrospira platensis algae.

The cosmetic composition according to the present disclosure has intensive antiwrinkle properties making it possible to combat deep wrinkles, skin slackening and a dull complexion.

The cosmetic composition according to the disclosure is rich in antioxidant compounds. It acts against free radicals and stimulates cellular oxygenation. With continued application, the contour of the face is resculpted, deep wrinkles are reduced and the complexion is more uniform. The cosmetic composition according to the disclosure also makes it possible to reduce bags and dark circles under the eyes and protects from dehydration for improved comfort.

The cosmetic composition according to the disclosure also has anti-mark effects and stimulates cellular detoxification, protects from the effects of the sun and pollution, and increases the ability to combat marks characterized by an excess of melanin.

Depending on the intensity of the marks, two to four applications daily are necessary. Light and diffuse marks disappear within a few weeks, while dark marks with a defined outline may require 6 to 12 months to disappear completely. The cosmetic composition according to the disclosure is also recommended for residual marks from heavy acne breakouts. Moreover, it has a moisturizing effect for a supple and soft skin.

At the end of the treatment of the biomass according to the method of the disclosure, said biomass enriched in phycobiliproteins may be filtered and washed. It may be incorporated into the draining and drying circuit of the production line. It may be sold in para-pharmaceutical formulations, such as, for example, food supplements intended to reinforce the immune system in infants or cosmetic products with strong antioxidant power.

The biomass may also serve for the extraction and clarification of one or more phycobiliproteins. For example, the phycocyanin produced may be concentrated and stabilized and thus be introduced into food, cosmetic or medical formulations.

The disclosure also relates to a dermatological composition containing, as active substance, a biomass or a mixture of biomasses of photosynthetic microalgae, in particular of cyanobacteria, more particularly of Arthrospira platensis, having a content of phycobiliproteins at least equal to 20% of the dry weight of said biomass, as defined above, and a pharmaceutically acceptable carrier.

According to embodiments, the biomass may have a content of phycobiliproteins at least equal to 21%, 22%, 23%, 24% or 25% of the dry weight of said biomass.

According to embodiments, the biomass may have a content of phycobiliproteins of from 20% to 25% of the dry weight of said biomass.

Said photosynthetic microalgae are in particular chosen from Rhodophyceae, Cryptophyceae and cyanobacteria, more particularly cyanobacteria, even more particularly Arthrospira platensis and Aphanizomenon flos-aquae, especially Klamath alga.

The disclosure also relates to the use of a biomass or a mixture of biomasses of photosynthetic microalgae, in particular of cyanobacteria, more particularly of Arthrospira platensis, having a content of phycobiliproteins at least equal to 20% of the dry weight of said biomass, as defined above, as antioxidant.

According to embodiments, the biomass may have a content of phycobiliproteins at least equal to 21%, 22%, 23%, 24% or 25% of the dry weight of said biomass.

According to embodiments, the biomass may have a content of phycobiliproteins of from 20% to 25% of the dry weight of said biomass.

Said photosynthetic microalgae are in particular chosen from Rhodophyceae, Cryptophyceae and cyanobacteria, more particularly cyanobacteria, even more particularly Arthrospira platensis and Aphanizomenon flos-aquae, especially Klamath alga.

### DESCRIPTION OF THE TABLES AND FIGURES

Table 1 represents the composition of an induction solution which may be used within the context of the disclosure, comprising 4 g/l of NaNO₃.

Table 2 represents the experimental matrix: the combination of the three levels corresponding to two factors, brightness and nitrogen concentration, and also the phycobiliprotein yield obtained expressed as percentage of dry weight.

FIG. 1 represents the iso yield curves of the phycocyanin synthesis as a function of the light intensity (μmol m⁻² s⁻¹) and of the nitrogen concentration (g/l) for an induction duration of 3 hours.

FIG. 2 represents the iso yield curves of the phycocyanin synthesis as a function of the light intensity (μmol m⁻² s⁻¹) and of the nitrogen concentration (g/l) for an induction duration of 24 hours.

FIG. 3 represents the iso yield curves of the phycocyanin synthesis as a function of the light intensity (μmol m⁻² s⁻¹) and of the nitrogen concentration (g/l) for an induction duration of 3 days.

### EXAMPLES

### Example 1

### Phycocyanin Synthesis

The variation in the phycocyanin synthesis yield is studied for various nitrogen concentrations and at various light intensities.

The study is carried out according to the methodology of experimental designs by way of a three-level factorial design with interaction (Goupy, 2001).

The fresh biomass is obtained from a culture, in the exponential phase, of the photosynthetic cyanobacterium Arthrospira platensis, concentrated 5 times. The induction solution used is modified Zarrouk's medium: concentration of NaNO₃ as nitrogen source of 1 to 4 g/l. The biomass concentrated in the induction medium is subjected to various light intensities within a range of between 10 to 50 μmol m⁻² s⁻¹.

Eleven experiments are carried out, nine of which represent the combination between the three levels corresponding to the two factors (minimum, maximum and middle). The three others represent the central points and make it possible to evaluate the experimental error. The experimental matrix is represented in table 2. For each condition, three samples are taken at different time periods: 3 hours, one day and 3 days of treatment.

The phycocyanin is extracted at the end of each of the 11 experimental conditions. The extraction is repeated twice so as to extract all the phycocyanin. Then, the phycocyanin yield (percentage of phycocyanin per gram of dry matter) is calculated.

The phycocyanin synthesis yield (Ren Phy) is modeled as a function of the NaNO₃ nitrogen concentration (N) and the light intensity (Lum).

The various treatments show that induction of 3 hours gives the highest yields. The model obtained under these conditions is expressed by the following equation:

RenPhy%⁽²⁾=65−45*Lum+458*N+1.02*Lum²−51*N²−3.14*Lum*N

The model is valid at 95%, with a correlation coefficient (R²) of 95%.

The standard error is 15%.

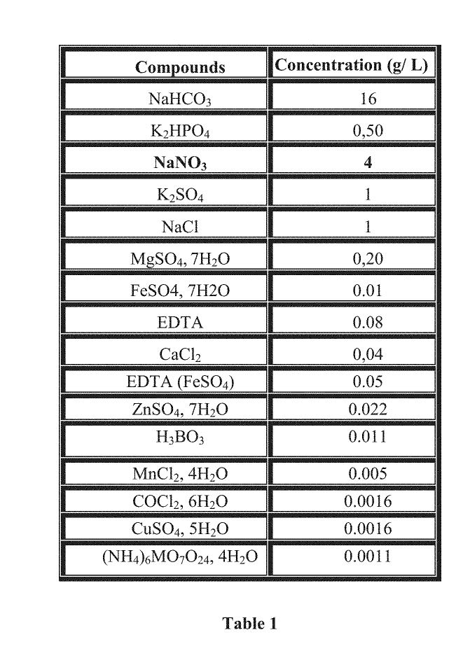

The iso yield curves of the phycocyanin synthesis as a function of light intensity (μmol m⁻² s⁻¹) and NaNO₃ concentration (g/l) and obtained after 3 hours of treatment according to the method of the present disclosure, are illustrated in FIG. 1. A light intensity of 10 μmol m⁻² s⁻¹ and an NaNO₃ concentration of 4 g/l makes it possible to obtain a phycocyanin yield of 22% to 24% per gram of dry matter.

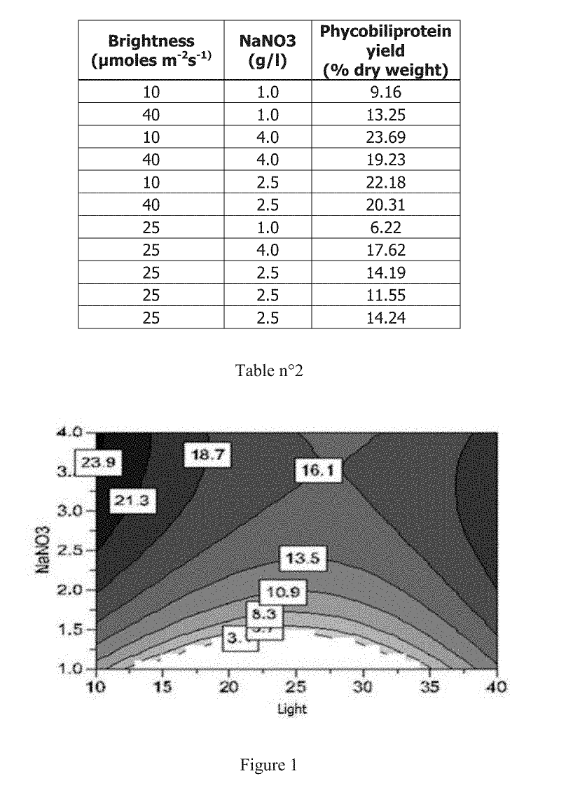

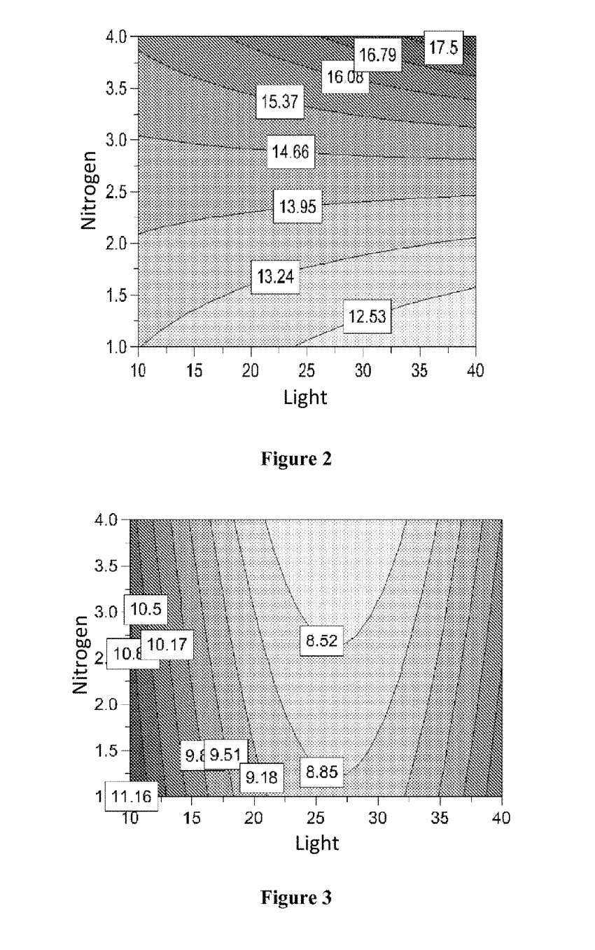

When the induction of the synthesis is prolonged to 1 day, the phycocyanin yields do not exceed a maximum of 18% (FIG. 2). Treatment of 3 days only makes it possible to obtain a maximum yield of less than 12% (FIG. 3).

### Example 2

### Antiwrinkle Cream

An antiwrinkle cream is obtained with the following composition:

Water: 59%,

Sweet almond oil: 18%,

Phycobiliproteins dissolved in glycerin: 7%,

Palm oil: 5%,

Castor oil: 3%,

Algae extract: 3% (beta-carotene),

Beeswax: 2%,

Xanthan gum: 1.7%,

Fragrance: 1%,

Ethylparaben: 0.1%,

Methylparaben: 0.1%,

Benzylparaben: 0.1%.

### Example 3

### Anti-Mark Cream

An anti-mark cream is obtained with the following composition:

Anti-mark cream:

Water: 59%,

Sweet almond oil: 19.5%,

Phycobiliproteins dissolved in glycerin: 7%,

Palm oil: 5%,

Castor oil: 3%,

Algae extract: 1.5% (beta-carotene),

Beeswax: 2%,

Xanthan gum: 1.7%,

Fragrance: 1%,

Ethylparaben: 0.1%,

Methylparaben: 0.1%,

Benzylparaben: 0.1%.

### REFERENCES

Erikson N. T, 2008, production of phycocyanin, a pigment with applications in biology, biotechnology, foods and medicine. Appl Microbiol Biotechnol, 80, 1-14.

Hemlata T. F, 2009, Screening of cyanobacteria for phycobiliproteins and effect of different environmental stress on its yield. Bull Environ Contam Toxicol, 83, 509-515.

Liu Y, Xu L, Cheng N, Lin L, Zheng Ch, 2000, Inhibitory effect of phycocyanin from Arthrospira platensis on the growth of human leukemia K562 cells. Journal of applied phycology, 12, 125-130.

Chen F, Zhang Y and Guo S, 1996. Growth and phycocyanin formation of Spirulina platensis in photoheterotrophic culture. Biotechnology Letters, 18, Issue 5, pp 603-608.

Remziye Aysun Kepekçi & Saadet Demirörs Saygideger, 2012. Enhancement of phenolic compound production in Spirulina Platensis by two-step batch mode cultivation. J Appl Phycol, 24:897-905.

Goupy, J., 2001, Introduction aux plans d′experiences, [Introduction to experimental designs] second Edition, Dunod.

1. A method for preparing a biomass of photosynthetic microaigae, having a content of phycobiliproteins at least equal to 20% of the dry weight of said biomass, comprising inducing the synthesis of phycobiliproteins in a biomass, the growth of which is blocked.

2. The method as claimed in claim 1, characterized in that the growth of said biomass of microalgae is blocked by concentrating said biomass in an induction tank, said concentration of said biomass in said tank being from 3 to 13 times higher than the density enabling the optimum growth of microalgae in culture, i.e. 0.4 g/l.

3. The method as claimed in claim 1, wherein said concentration of said biomass is from 1 g/l to 5 g/l.

4. The method as claimed in claim 1, wherein the inducing the synthesis of phycobiliproteins comprises:
exposing the biomass of photosynthetic microalgae to light flux, said flux having a light intensity of a value from substantially equal to or greater than 10 micromoles m⁻² s⁻¹ (μmol m⁻² s⁻¹) to a value substantially equal to or less than 13 μmol m⁻² s⁻¹
adding a source of nitrogen so as to obtain a concentration of NaNO₃ in said biomass of greater than 2.5 g/l of NaNO₃.

5. The method as claimed in claim 4, wherein the induction step is carried out for a duration of 3 hours, 4 hours or 5 hours.

6. The method as claimed in claim 1 for preparing a biomass of photosynthetic microalgae having a content of phycobiliproteins at least equal to 20% of the dry weight of said biomass, comprising the following steps:
a) blocking the growth of said biomass
b) inducing the synthesis of phycobiliproteins.

7. The method for preparing a biomass of photosynthetic microalgae as claimed in claim 6, comprising a prior step of collecting a biomass of microalgae.

8. The method as claimed in claim 6, comprising a step of collecting a biomass enriched in phycobiliproteins.

9. The method as claimed in claim 1, comprising the following steps:
a) collecting a biomass of microalgae
b) blocking the growth of said biomass
c) inducing the synthesis of phycobiliproteins
d) collecting said biomass enriched in phycobiliproteins.

10. The method as claimed in 1, in which the photosynthetic microalgae are chosen from one of cyanobacteria, rhodophytes and cryptophytes.

11. The method as claimed in one of the preceding claims, in which the photosynthetic microalga is chosen from Rhodophyceae, Cryptophyceae and cyanobacteria.

12. The method as claimed in claim 1, characterized in that the phycobiliproteins are chosen from phycocyanin, allophycocyanin, phycoerythrin or phycoerythrocyanin, or a mixture of at least two of these phycobiliproteins.

13. The method as claimed in claim 4, in which the nitrogen is provided by a culture medium comprising a final concentration of greater than 2.5 g/l and less than 5 g/l of NaNO₃.

14. A biomass of photosynthetic microalgae having a content of phycobiliproteins at least equal to 20% of the dry weight of said biomass, obtained by the method of claim 1.

15. The biomass as claimed in claim 14, extracted from Arthrospira platensis algae.

16. The use of a biomass of photosynthetic microalgae obtained by the method of claim 1, having a content of phycobiliproteins at least equal to 20% of the dry weight of said biomass, as a food supplement or a constituent of a cosmetic, dermatological or nutraceutical preparation.

17. A cosmetic, dermatological, or nutraceutical composition or a composition intended as a food supplement, prepared from a biomass of photosynthetic microalgae having a content of phycobiliproteins at least equal to 20% of the dry weight of said biomass.

18. The cosmetic, dermatological, or nutraceutical composition or the composition intended as a food supplement, as claimed in claim 17, prepared from a biomass of Arthrospira platensis algae.

19. The method for preparing a biomass of photosynthetic microalgae according to claim 1, wherein the biomass of photosynthetic microalgae is a cyanobacteria.

20. The method of claim 11, wherein the photosynthetic microalga is chosen from cyanobacteria.

21. The method of claim 11, wherein the cyanobacteria is chosen from Arthrospira platensis and Aphanizomenon flos-aquae.

22. The method of claim 21, wherein the cyanobacteria is Klamath alga.
